(19) United States
(12) Patent Application Publication
(10) Pub. No.: US 2025/0380936 A1
(43) Pub. Date: Dec. 18, 2025

(54) DISSOLVABLE INSERT FOR DELIVERY CATHETER FOR IMPLANTABLE MEDICAL DEVICES

(71) Applicant: Medtronic, Inc., Minneapolis, MN (US)

(72) Inventors: Elliot C. Schmidt, Minneapolis, MN (US); Kathryn E. Hilpisch, Cottage Grove, MN (US); Gregory N. Nesseth, Forest Lake, MN (US)

(21) Appl. No.: 19/235,213

(22) Filed: Jun. 11, 2025

Related U.S. Application Data
(60) Provisional application No. 63/659,036, filed on Jun. 12, 2024.

(51) Int. Cl. A61B 17/00

(52) U.S. Cl. CPC A61B 17/0057; A61B 2017/00623

(57) ABSTRACT

A medical device system comprising: an implantable medical device (IMD); a delivery catheter extending from a proximal end to a distal end, wherein the distal end of the delivery catheter defines a receptacle sized to retain the IMD, the receptacle defining a distal opening; and a dissolvable insert configured to enclose the distal opening of the receptacle, wherein the dissolvable insert is configured to at least partially dissolve within a body fluid of a patient to enable deployment of the IMD from within the receptacle of the delivery catheter.

### RELATED APPLICATIONS

This application claims the benefit of U.S. Provisional Application Ser. No. 63/659,036, filed Jun. 12, 2024, the entire contents of which are incorporated herein by reference.

### TECHNICAL FIELD

The present invention pertains to implantable medical device systems, and more specifically, to components of delivery catheters for implantable medical devices.

### BACKGROUND

The cardiac conduction system includes the sinus atrial (SA) node, the atrioventricular (AV) node, the bundle of His, bundle branches and Purkinje fibers. A heartbeat is initiated in the SA node, which may be described as the natural “pacemaker” of the heart. An electrical impulse arising from the SA node causes the atrial myocardium to contract. The electrical impulse, or electrical pulse or signal, is conducted to the ventricles via the AV node which inherently delays the conduction to allow the atria to stop contracting before the ventricles begin contracting thereby providing proper AV synchrony. The electrical impulse is conducted from the AV node to the ventricular myocardium via the bundle of His, bundle branches, and Purkinje fibers.

The leadless pacemaker, which is significantly smaller than a conventional pacemaker coupled to one or more transvenous leads, is a self-contained generator and electrode system configured to be implanted directly into the heart. The leadless pacemaker, which does not utilize leads extend from out of the heart of a patient, eliminates several complications associated with transvenous pacemakers and leads such as, for example, pocket infections, hematoma, lead dislodgment, and lead fracture. The leadless pacemaker also has cosmetic appeal because there is no chest incision or visible pacemaker pocket.

The leadless pacemaker includes one or more electrodes on its outer housing to deliver therapeutic electrical signals and/or sense intrinsic depolarizations of the heart. Intracardiac medical devices may provide cardiac therapy functionality, such as sensing and pacing, and may also be used to treat either atrial or ventricular arrhythmias or fibrillation.

The leadless pacemaker device may be implanted via a femoral vein transcatheter approach and requires no chest incision or subcutaneous generator pocket. The catheter system utilized to deploy the leadless pacemaker includes a distal end with a delivery cup housing the self-contained generator and electrode system, referred to herein as a pacing capsule. The delivery cup is maneuvered into the proper position, e.g., in the right atrium at or near the triangle of Koch (TOK) or the right ventricle near the apex or interventricular septum, under fluoroscopic visualization or using a sonogram produced by an ultrasound imaging system.

### SUMMARY

A delivery catheter of a medical device system may include a delivery cup at a distal end that is configured to retain an implantable medical device (IMD). The delivery catheter may retain the IMD within the delivery cup as the clinician navigates the delivery catheter within vasculature of a patient to a target site within the patient. Once the distal end of the delivery catheter is at the target site, the clinician may advance the IMD out of the delivery cup and affix the IMD to tissue at or around the target site. In some examples, the target site is located in the heart of the patient (e.g., in a chamber of the heart of the patient).

As the clinician advances the delivery catheter within the vasculature of the patient, the distal end of the delivery catheter may contact wall tissue of one or more blood vessels of the patient, which may lead to unintended perforation of the tissue and/or difficulty in navigating the delivery catheter through the one or more blood vessels and/or within a heart of the patient(e.g., around one or more turns in the one or more blood vessels and/or around one or more features within the heart).

The devices, systems, and techniques discussed herein describe a dissolvable insert configured to be placed at least partially over and/or at least partially within the delivery cup of the delivery catheter. The dissolvable insert may enclose an inner volume of the delivery cup, e.g., to isolate the IMD from tissue of the patient and inhibit unintended contact between the IMD and the tissue as the clinician advances the delivery catheter within the vasculature. The dissolvable insert may define a distal atraumatic tip for the delivery catheter, which may reduce a risk of unintended perforation of the tissue by the delivery catheter as the clinician navigates the delivery catheter through the vasculature. The dissolvable insert may improve the capability of the distal end of the delivery catheter to navigate within the vasculature, e.g., around turn(s) in the vasculature.

The dissolvable insert may be formed from one or more biocompatible materials that may dissolve over time within the body of the patient (e.g., within blood of the patient). Once the dissolvable insert is at least partially dissolved, the clinician may advance the IMD out of the delivery cup and affix the IMD to the tissue at the target site. Use of a dissolvable insert with a delivery catheter may eliminate a need for a feature for releasing and retaining a non-dissolvable insert by the delivery catheter or another element of the medical device system, which may simplify the structure of the delivery catheter and simplify the IMD implantation process.

While the dissolvable insert is primarily described herein with respect to a delivery catheter configured to navigate within the vasculature of the patient (e.g., to a heart of a patient), the dissolvable insert may be coupled to another delivery catheter configured to navigate within another body lumen of the patient.

In some examples, this disclosure describes a medical device system comprising: an implantable medical device (IMD); a delivery catheter extending from a proximal end to a distal end, wherein the distal end of the delivery catheter defines a receptacle sized to retain the IMD, the receptacle defining a distal opening; and a dissolvable insert configured to enclose the distal opening of the receptacle, wherein the dissolvable insert is configured to at least partially dissolve within a body fluid of a patient to enable deployment of the IMD from within the receptacle of the delivery catheter.

In some examples, this disclosure describes a dissolvable insert comprising: a distal portion defining a first outer diameter; and a proximal portion connected to the distal portion and defining a second outer diameter, the second outer diameter being different from the first outer diameter, wherein the dissolvable insert is configured to interface with a receptacle of a catheter to at least partially enclose an inner volume of the receptacle, wherein the proximal portion is sized to enter the inner volume of the receptacle, and wherein the distal portion is sized to remain outside of the inner volume of the receptacle, and wherein the dissolvable insert is configured to at least partially dissolve within a body fluid.

In some examples, this disclosure describes a method comprising: disposing an implantable medical device (IMD) within an inner volume of a receptacle at a distal end of a delivery catheter of the medical device system via a distal opening of the receptacle; and securing a dissolvable insert to the distal end of the delivery catheter and over the distal opening of the receptacle to at least partially enclose the inner volume of the receptacle, wherein the dissolvable insert is configured to at least partially dissolve within a body fluid and allow access to the inner volume of the receptacle.

In some examples, this disclosure describes a method comprising: advancing, via a delivery catheter of a medical device system, an implantable medical device (IMD) of the medical device system through vasculature of a patient to a target location within the patient, wherein the distal end of the delivery catheter defines a receptacle, wherein the IMD is disposed within an inner volume of the receptacle, and wherein the medical device system further comprises a dissolvable insert disposed over a distal opening of the receptacle to retain the IMD within the receptacle; releasing the dissolvable insert from the receptacle; advancing the IMD distally from within the inner volume of the receptacle; and affixing the IMD to tissue of the patient at the target location.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram showing potential implant sites for embodiments of the present invention.

FIG. 2 is a conceptual diagram of an example medical device system with an implantable medical device (IMD) loaded in a delivery catheter.

FIG. 3 is conceptual diagram illustrating the example IMD of FIG. 2 in a deployed configuration.

FIG. 4A is a conceptual diagram illustrating a top view of an example dissolvable insert of the medical device system of FIG. 2.

FIG. 4B is a conceptual diagram illustrating a bottom view of the example dissolvable insert of FIG. 4A.

FIG. 4C is a cross-sectional diagram illustrating a cross-sectional view of the dissolvable insert of FIG. 4A, the cross-section being taken along line A-A of FIG. 4A.

FIG. 5A is a conceptual diagram illustrating a top view of an example dissolvable insert of the medical device system of FIG. 2.

FIG. 5B is a conceptual diagram illustrating a bottom view of the example dissolvable insert of FIG. 5A.

FIG. 5C is a cross-sectional diagram illustrating a cross-sectional view of the dissolvable insert of FIG. 5A, the cross-section being taken along line B-B of FIG. 5A.

FIG. 6A is a conceptual diagram illustrating an example dissolvable insert of the medical device system of FIG. 2.

FIG. 6B is a conceptual diagram illustrating an example dissolvable insert of the medical device system of FIG. 2.

FIG. 6C is a conceptual diagram illustrating an example dissolvable insert of the medical device system of FIG. 2.

FIG. 7 is a flow chart illustrating an example technique of assembling the medical device system of FIG. 2.

FIG. 8 is a flow chart illustrating an example technique of implanting a medical device via the medical device system of FIG. 2.

Like symbols in the drawings indicate like elements.

### DETAILED DESCRIPTION

A clinician may implant an implantable medical device (IMD) to tissue at a target site within a patient via a delivery catheter. The delivery catheter may include a delivery cup disposed at or around a distal end of the delivery catheter. The delivery cup may define an inner volume sized to retain the IMD (e.g., in a collapsed configuration). During the implantation procedure, the clinician may advance the delivery catheter containing the IMD within the delivery cup within the patient until a distal end of the delivery catheter reaches the target site in the patient. The clinician may then advance the IMD from within the IMD to tissue at the target site and affix the IMD to the tissue (e.g., via one or more fixation helices, tines, barbs, or the like).

The delivery cup may define a distal opening at or around the distal end of the delivery cup. The distal opening may be at or around the distal end of the delivery catheter. The delivery cup may retain a distal portion of a tether assembly of a medical device system. The tether assembly may be coupled to a proximal end of the IMD. The clinician may advance the IMD from within the delivery cup and/or retract the IMD into the delivery cup via the tether assembly. The IMD may enter or exit the inner volume of the delivery cup via the distal opening of the delivery cup.

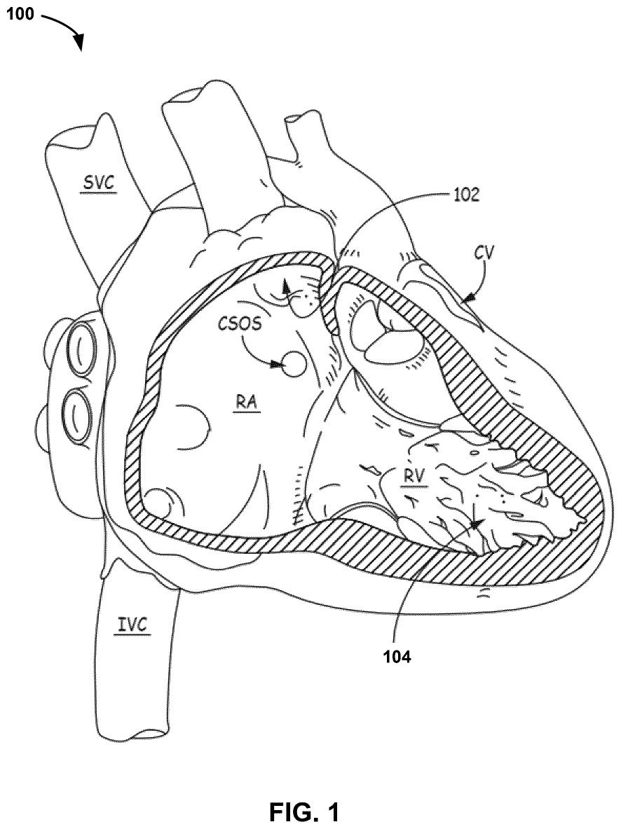

FIG. 1 is a conceptual diagram showing potential target sites for embodiments of the present invention. While FIG. 1 illustrates potential target sites within heart 100 of the patient, other potential target sites may be in other locations within the body of the patient, e.g., at other locations within the cardiovascular system of the patient. As illustrated in FIG. 1, potential target sites for an IMD within heart 100 may be within appendage 102 of the right atrium, in proximity to apex 104 of the right ventricle, at or around a triangle of Koch of heart 100, or the like.

While the delivery catheter is primarily described in this disclosure as being configured to be navigated within blood vessels of a vasculature of the patient, the devices, techniques, and methods described herein may be applied to delivery catheters configured for use within one or more other body lumen of the patient.

In some examples, as the clinician navigates the delivery catheter within the vasculature of the patient, the distal end of the delivery catheter may come into contact with blood vessel wall tissue of the patient. The contact between the delivery catheter and the wall tissue may lead to unintended perforation of the tissue by the distal end of the delivery catheter. For example, the distal end of the delivery cup may cause an unintended perforation of the tissue. In some examples, there may be unintended advancement of a distal end of the IMD out of the delivery cup as the clinician advances the delivery catheter within the vasculature, which may lead to unintended deployment of the IMD and/or unintended perforation of wall tissue of a blood vessel by the IMD. The unintended perforations may require the clinician to reposition the delivery catheter within a blood vessel and/or may increase the complexity of navigating the delivery catheter within the vasculature, e.g., around a bend in the vasculature.

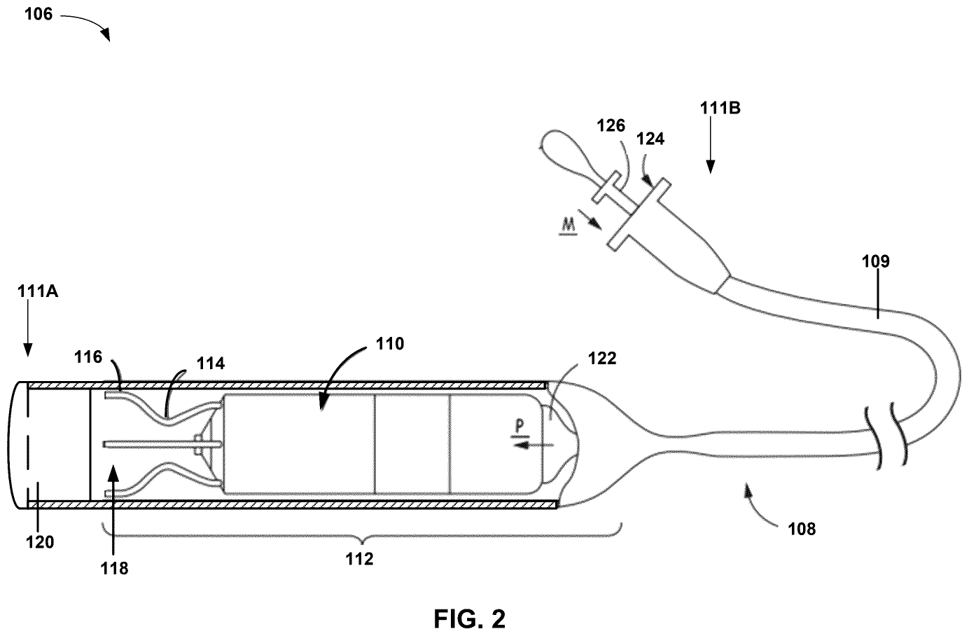

FIG. 2 is a conceptual diagram of an example medical device system 106 (alternatively referred to herein as “system 106”) with an IMD 110 loaded in a delivery catheter 108. The clinician may deliver IMD 110 to a target site via delivery catheter 108 of system 106. IMD 110 may be an implantable cardiac device such as, but is not limited to, an implantable pacemaker, an implantable cardiac monitor, an implantable cardioverter defibrillator, or the like. The clinician may navigate a distal end 111 of delivery catheter 108 through the vasculature of the patient to the implant site.

As illustrated in FIG. 2, delivery catheter 108 may include an elongated body 109 extending from distal end 111A to proximal end 111B of delivery catheter 108. Elongated body 109 may define and/or may be attached to delivery cup 112 at or around distal end 111A. For example, as illustrated in FIG. 2, a distal end of delivery cup 112 may define distal end 111A. Delivery cup 112 may define inner volume 118 configured to retain IMD 110.

IMD 110 may be loaded into delivery cup 112 in a delivery configuration. In the delivery configuration, one or more fixation features 114 may be held in the deployed configuration. Fixation features 114 may include, but are not limited to, barbs, tines, helices, or the like. In the deployed configuration, distal ends 116 of fixation features 114 may extend distally towards distal end 111A.

A tether assembly 122 may be removably connected to a proximal end of IMD 110. Tether assembly 122 may extend through delivery catheter 108 through an inner lumen within elongated body 109. A proximal end of tether assembly 122 may extend proximally from a proximal opening 124 of delivery catheter 108. The clinician may push tether assembly 122 (e.g., via a proximal handle 126 of tether assembly 122) along direction M to advance IMD 110 out of delivery cup 112 along direction P, and vice versa.

A dissolvable insert 120 may be coupled to delivery catheter 108 to enclose inner volume 118 of delivery cup 112. At least a portion of dissolvable insert 120 may define distal end 111A of delivery catheter 108. In some examples, as illustrated in FIG. 2, at least a portion of dissolvable insert 120 may be disposed within inner volume 118. When dissolvable insert 120 is coupled to delivery catheter 108, dissolvable insert 120 may be separated from IMD 110, e.g., such that distal end 118 of fixation features 114 do not contact dissolvable insert 120 within delivery cup 112.

Dissolvable insert 120 may define an atraumatic tip at or around distal end 111A of delivery catheter 108, e.g., to inhibit unintended perforation of tissue by the distal end of delivery cup 112 and/or by distal end 118 of fixation feature 114. Dissolvable insert 120 may facilitate retention of IMD 110 within delivery cup 112, e.g., by inhibit unintended advancement of IMD 110 out of delivery cup 112 in direction P.

Dissolvable insert 120 may be formed from a dissolvable material (e.g., a sugar). Dissolvable insert 120 may dissolve when disposed within a body fluid of the patient (e.g., blood of the patient). The ability of dissolvable insert 120 to dissolve within the body fluid of the patient may allows for access to inner volume 118 when distal end 111A of delivery catheter 108 is at or around a target site without requiring the removal and retention of an insert for delivery cup 112. When dissolvable insert 120 is at least partially dissolved, the clinician may advance IMD 110 out of the distal opening of delivery cup 112 in direction P to puncture tissue at the target site and affix IMD 110 to the tissue at the target site. In some examples, when dissolvable insert 120 is partially dissolved, the clinician may decouple a remaining portion of dissolvable insert 120 prior to advancing IMD 110 via injection of a fluid (e.g., a contrast fluid) through delivery catheter 108 (e.g., through the inner lumen of elongated body 109 and into inner volume 118).

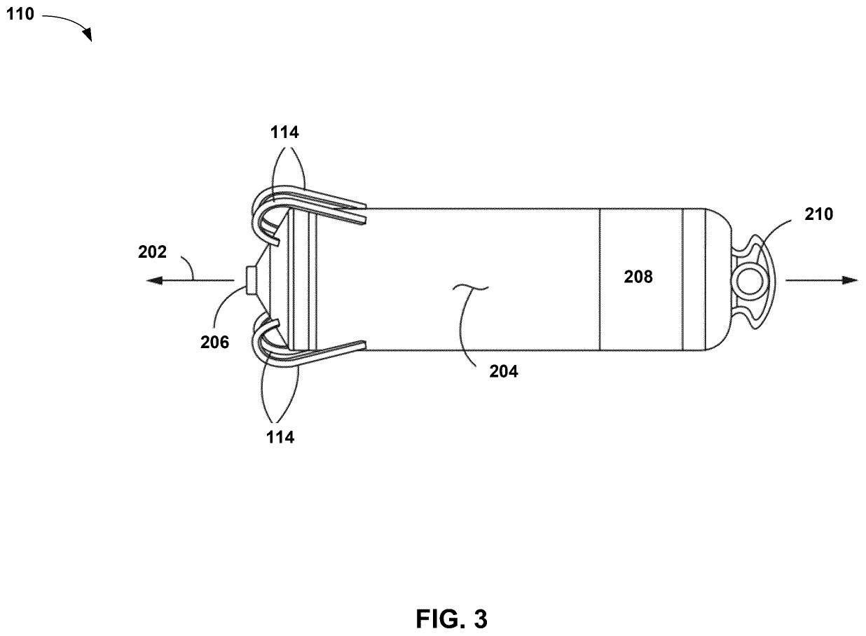

FIG. 3 is conceptual diagram illustrating IMD 110 of FIG. 2 in a deployed configuration. IMD 110 may include housing 204, electrode 206, electrode 208, and fixation features 114.

Housing 204 may be configured to retain a power source and one or more computing elements of IMD 110. Housing 204 may include an elongated body extending along longitudinal axis 202. IMD 110 may include electrodes 206, 208, and/or one or more other electrodes (e.g., electrically active portions of fixation features 114) which may form electrode pairs for the delivery of electrical stimulation signals (e.g., cardiac pacing signals) to the target site and/or the sensing of signals from the target site. Electrode 208 may be disposed on housing 202 and/or may be defined by an electrically active portion of housing 204.

Electrode 206 may be configured to contact the tissue of the patient at the target site. In some examples, as illustrated in FIG. 3, electrode 206 may be configured to contact the tissue without penetration of the tissue. In some examples, electrode 206 is configured to penetrate the tissue, e.g., to deliver and/or sense signals to a target site within the tissue and/or to affix IMD 110 to the tissue. For example, electrode 206 may be a helical electrode.

Fixation features 114 may transition between a collapsed configuration and a delivery configuration to affix IMD 110 to the tissue. In some examples, fixation features 114 are formed from a shape memory material (e.g., Nitinol) which, when unconstrained by delivery cup 112, expand radially outwards from the collapsed configuration to the delivery configuration to puncture the tissue. In some examples, one or more of fixation features 113 may define one or more electrically active regions. The one or more electrically active regions may be configured to operate as electrodes alongside and/or instead of one or more of electrodes 206, 208.

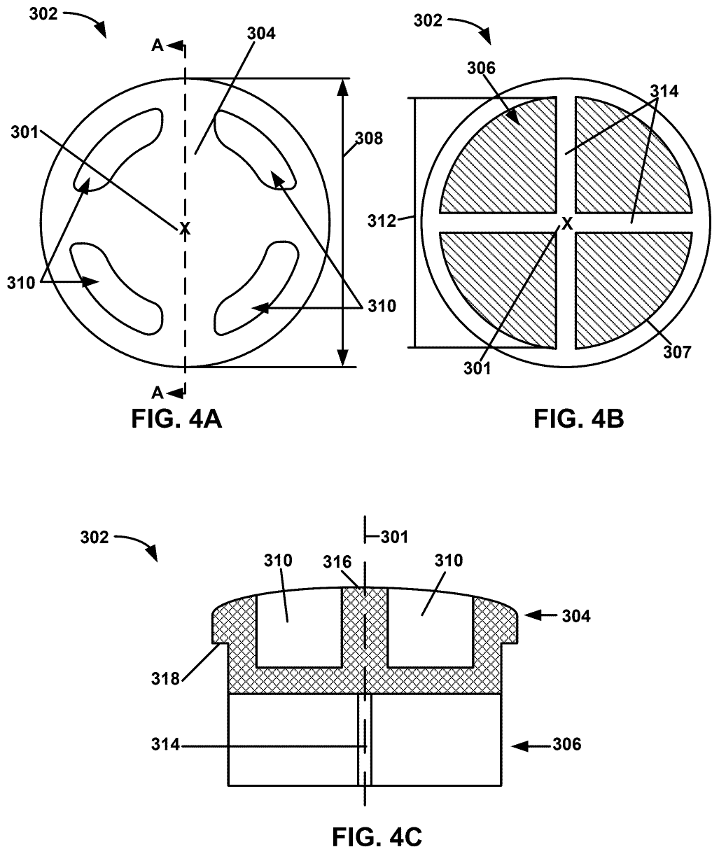

FIG. 4A is a conceptual diagram illustrating a top view of an example dissolvable insert 302 of the system 106 of FIG. 2. FIG. 4B is a conceptual diagram illustrating a bottom view of dissolvable insert 302 of FIG. 4A. FIG. 4C is a cross-sectional diagram illustrating a cross-sectional view of dissolvable insert 302 of FIG. 4A, the cross-section being taken along line A-A of FIG. 4A. As illustrated in FIGS. 4A-4C, dissolvable insert 302 may extend along longitudinal axis 301 and may define a distal portion 304 and a proximal portion 306 coupled to distal portion 304. Dissolvable insert 302 may be an example of dissolvable insert 120, as illustrated in FIG. 2.

Distal portion 304 may define a width 308 (alternatively referred to herein as diameter 308) and one or more recesses 310 on outer surface 316 of distal portion 304. Distal portion 304 may define a circular shape, a triangular shape, a rectangular shape, or another polygonal shape. Each of recesses 310 may extend at least partially from the outer surface of distal portion 304 in a proximal direction along longitudinal axis 301. In some examples, each of recesses 310 extend completely from the outer surface 316 of dissolvable insert 302 to a proximal end of dissolvable insert 302. Each of recesses 310 may allow for a body fluid to enter and/or exit the respective recess 310. In such examples, each recess 310 may increase a contact surface area between dissolvable insert 302 and a body fluid when dissolvable insert 302 is coupled to delivery catheter 108 and disposed within the body of the patient. The increase in contact surface area may correspond to a reduction in an amount of time required to fully dissolve or dissolve a threshold portion of dissolvable insert 302 (e.g., to dissolve 50%, 75%, 90% of dissolvable insert 302).

Dissolvable insert 302 may include one, two, or three or more recesses 310. The dimensions of recesses 310 may be based at least in part on an intended increase in contact surface area provided by recesses 310. For example, a larger intended increase in contact surface area for dissolvable insert 302 may correspond to an increase in the number of recesses 310, the width of recesses 310, the length of recesses 310, and/or the depth of recesses 310, and vice versa.

Recesses 310 may be sized (e.g., relative to a surface area of dissolvable insert 302) to control a rate of dissolution of dissolvable insert 302. The rate of dissolution for dissolvable insert 302 may be selected based at least in part on the location of the target site within the body of the patient, an incision site (e.g., into the vasculature) on the body of the patient, and/or an expected duration of the implantation procedure for IMD 110. The rate of dissolution for dissolvable insert 302 may be selected such that dissolvable insert 302 does not fully or substantially dissolve prior to distal end 111A of delivery catheter 108 reaching the target site.

In some examples, recesses 310 may facilitate the introduction of a fluid (e.g., a contrast fluid) from delivery catheter 108 into the vasculature of the patient. For example, the clinician may introduce the fluid into delivery cup 112 via the inner lumen of elongated body 109. The fluid may exit inner volume 118 of delivery cup 112 and into the vasculature via one or more of recesses 310. In some examples, where dissolvable insert 302 is at least partially dissolved, the introduction of the fluid may separate a remaining portion of dissolvable insert 302 from delivery catheter 108, e.g., to enable advancement of IMD 110 from inner volume 118 of delivery cup 112. Once dissolvable insert 302 is separated from delivery catheter 108, the remaining portion of dissolvable insert 302 will dissolve within the body fluid over time.

Proximal portion 306 of dissolvable insert 302 may include one or more protrusions 307 extending proximally along longitudinal axis 301 from distal portion 304. Protrusions 307 may define a width 312 (alternatively referred to herein as “diameter 312”) of proximal portion 306. Diameter 312 may be less than or equal to diameter 308. Diameter 312 may be less than, greater than, or equal to an inner diameter of delivery cup 112 of delivery catheter 108. In some examples, a radially outer surface of proximal portion 306 (e.g., as defined by protrusions 307) may contact and interface with an inner surface of delivery cup 112 to couple dissolvable insert 302 to delivery cup 112.

Distal portion 304 may define a step 318 from diameter 308 to diameter 312 to inhibit unintended overtravel of dissolvable insert 302 into delivery cup 112. In such examples, distal portion 304 may be flush with a distal end of delivery cup 112 or may be at least partially protruding from delivery cup 112.

Proximal portion 306 may define one or more channels 314 separating protrusions 307. Each channel 314 may extend along at least a portion of the longitudinal length of channels 307. In some examples, channels 314 may extend along a full longitudinal length of channels 307, as illustrated in FIG. 4C. Each of channels 314 may define a width greater than or equal to a width of one or more of fixation features 114 of IMD 110. When dissolvable insert 302 is coupled to delivery catheter 108, channels 314 may facilitate separation of dissolvable insert 302 from IMD 110, e.g., to reduce a likelihood of unintended contact between dissolvable insert 302 and IMD 110.

Dissolvable insert 302 may be formed from a biocompatible material capable of being dissolved within a body fluid (e.g., within blood). The material may include, but is not limited to, a sugar. A manufacturing assembly may form dissolvable insert 302 via a molding process (e.g., as a single component). In some examples, the manufacturing assembly may shape dissolvable insert 302 by removing material from a larger molded element. In such examples, the manufacturing assembly may remove material via one or more of a cutting tool or the like.

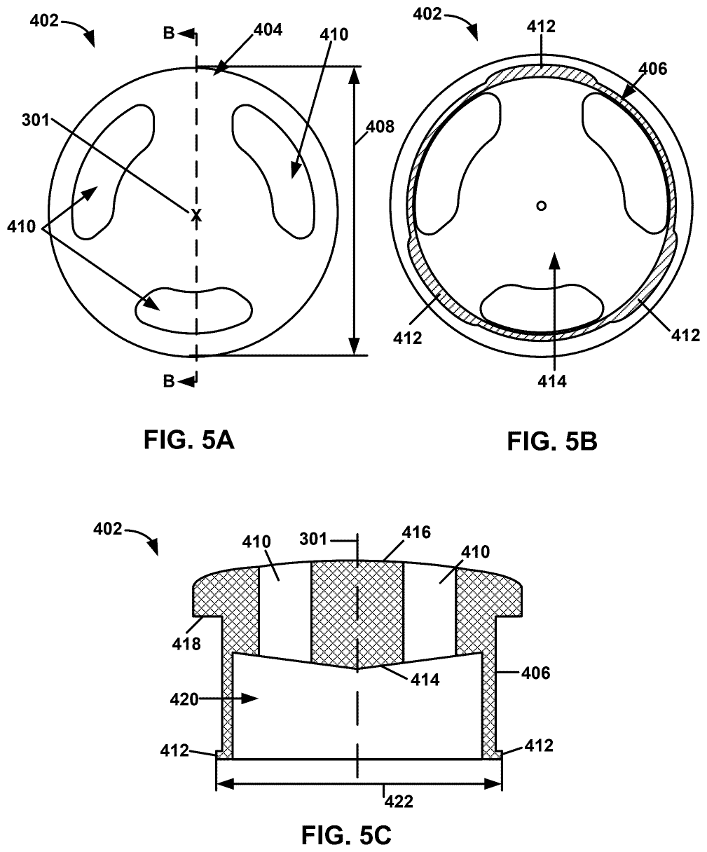

FIG. 5A is a conceptual diagram illustrating a top view of an example dissolvable insert 402 of system 106 of FIG. 2. FIG. 5B is a conceptual diagram illustrating a bottom view of dissolvable insert 402 of FIG. 5A. FIG. 5C is a cross-sectional diagram illustrating a cross-sectional view of dissolvable insert 402 of FIG. 5A, the cross-section being taken along line B-B of FIG. 5A. Dissolvable insert 402 may be an example of dissolvable insert 120, as illustrated in FIG. 2.

Dissolvable insert 402 may extend along longitudinal axis 301 and may define a distal portion 404 and a proximal portion 406. Proximal portion 406 may define an elongated annular structure defining an inner volume 420. Distal portion 404 may define a width 408 (alternatively referred to herein as “diameter 308”). Proximal portion 406 may define one or more extensions 412 extending radially away from longitudinal axis 301. Extensions 412 may define a maximum width 422 (alternatively referred to herein as “diameter 422”). Diameter 412 may be less than or equal to diameter 408. In some examples, diameter 408 is greater than or equal to an outer diameter of delivery cup 112. In some examples, diameter 422 is up to an inner diameter of delivery cup 112. Dissolvable insert 402 may define a step 418 indicating a transition between distal portion 404 and proximal portion 406. In some examples, step 418 defines a change in the outer diameter of dissolvable insert 402 (e.g., from diameter 408 to a reduced diameter such as diameter 422).

Distal portion 404 may define a circular shape, a triangular shape, a rectangular shape, or another polygonal shape. Distal portion 404 may define one or more recesses 410 extending from outer surface 416 of distal portion 404 towards or into inner volume 420 of proximal portion 406. Each of recesses 410 may by substantially similar to recesses 310, as previously described herein. Recesses 410 may extend through the body of dissolvable insert 404 into inner volume 420. In some examples, each of recesses 410 may be a blind recess extending from outer surface 416 towards inner volume 420 without reaching inner volume 420. Dissolvable insert 402 may include one, two, or three or more recesses 410. The dimensions of recesses 410 may be based at least in part on an intended increase in contact surface area provided by recesses 410. For example, a larger intended increase in contact surface area for dissolvable insert 402 may correspond to an increase in the number of recesses 410, the width of recesses 410, the length of recesses 410, and/or the depth of recesses 410, and vice versa.

Proximal portion 406 of dissolvable insert 402 may include one or more extensions 412 protruding from the outer surface of proximal portion 406 and extending away from longitudinal axis 301. When dissolvable insert 402 is at least partially disposed within delivery cup 112, extensions 412 may interface with an inner surface of delivery cup 112 to removably couple dissolvable insert 402 to delivery catheter 108. The dimensions of and/or number of extensions 412 may correspond to a ease of removability of dissolvable insert 402 from delivery cup 112. For example, an increase in the number of extensions 412, a thickness of extensions 412, and/or a width of extensions 412 may increase the strength of the fixation bond between dissolvable insert 402 and delivery catheter 108, and vice versa.

A proximal surface 414 of dissolvable insert 402 within inner volume 420 may define a curved or conical surface, e.g., extending proximally along longitudinal axis 301. The shape of proximal surface 414 may facilitate the flow of body fluid and/or a fluid introduced through delivery catheter 108 around dissolvable inert 402. In some examples, the shape of proximal surface 414 may increase a structural integrity of dissolvable insert 402, e.g., compared to another dissolvable insert 402 of substantially similar mass or volume without the curved or conical proximal surface 414.

Inner volume 420 may be configured to retain fixation features 114 of IMD 110 when IMD 110 is loaded into delivery cup 112. Inner volume 420 may be sized to retain at least a portion of fixation features 114 without contacting fixation features 114. The separation of dissolvable insert 402 from fixation features 114 may inhibit unintended contact between dissolvable insert 402 and fixation features 114 and reduce any unintended effects of dissolvable insert 402 on fixation features 114.

Dissolvable insert 402 may be formed from a biocompatible material capable of being dissolved within a body fluid (e.g., within blood). The material may include, but is not limited to, a sugar. A manufacturing assembly may form dissolvable insert 402 via a molding process (e.g., as a single component). In some examples, the manufacturing assembly may shape dissolvable insert 402 may removing material from a larger molded element. In such examples, the manufacturing assembly may remove material via one or more of a cutting tool or the like.

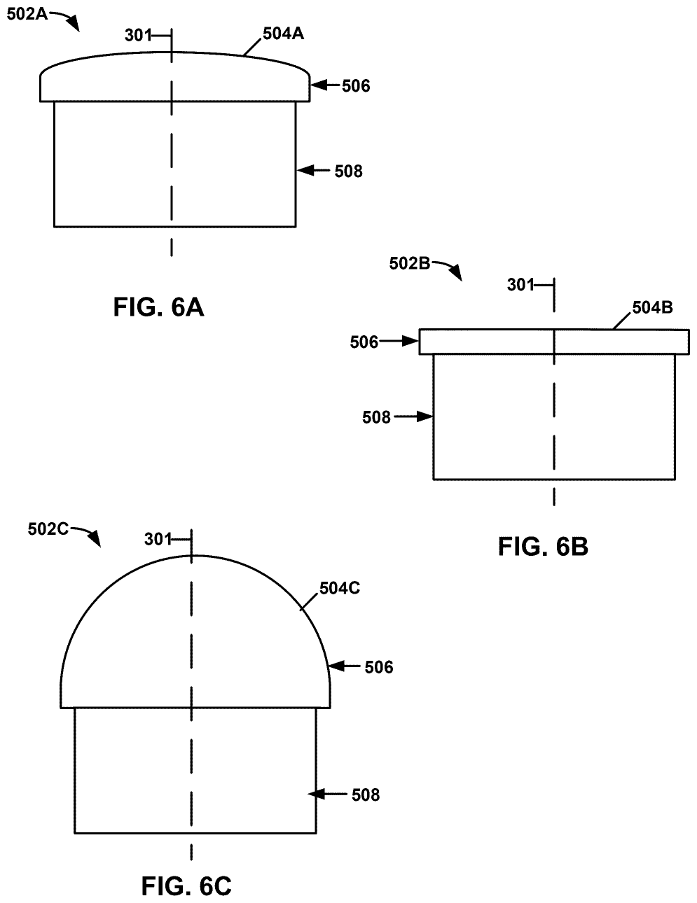

FIGS. 6A-6C are conceptual diagrams illustrating example dissolvable inserts 502A-502C (collectively referred to herein as “dissolvable inserts 502”) of system 106 of FIG. 2. Distal portion 506 of each of dissolvable inserts 502 may define outer surfaces 504A-C (collectively referred to herein as “dissolvable inserts 504”) with different shapes and/or structures. Proximal portions 508 of dissolvable inserts 502 may be substantially similar to other proximal portions of dissolvable inserts previously described herein (e.g., proximal portions 306, 406).

Outer surfaces 504 may define curved or rounded shape (e.g., as illustrated in FIG. 6A), a substantially flat shape (e.g., as illustrated in FIG. 6B), a hemispherical or domed shape (e.g., as illustrated in FIG. 6C), or any other blunted and/or atraumatic shape. The shape of outer surfaces 504 may be selected to inhibit unintended puncture of tissue of the patient by dissolvable inserts 502 as delivery catheter 108 and dissolvable insert 502 is navigated through the body of the patient. Distal portions 506 with differently shaped outer surfaces 504 may require different lengths of time to dissolve. For example, dissolvable insert 502C may require an increased amount of time to dissolve compared to dissolvable inserts 502A, 502B. Dissolvable inserts 502 with outer surfaces 504 of different shapes may be removably attached to delivery cup 112 of delivery catheter 108, e.g., based at least in part on the location of the target site and/or a distance between an incision cite on the patient and the target site.

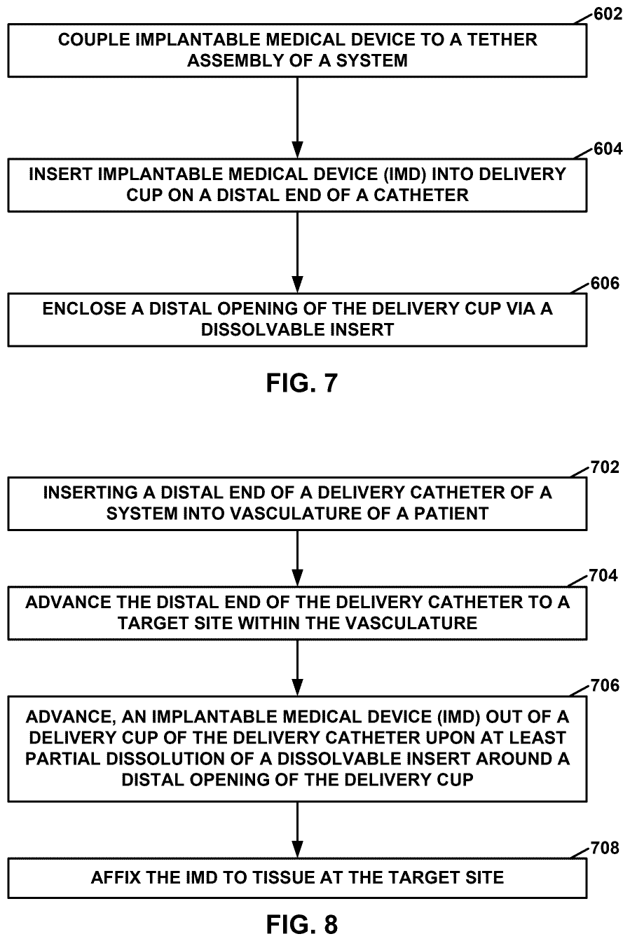

FIG. 7 is a flow chart illustrating an example technique of assembling system 106 of FIG. 2. While the technique illustrated in FIG. 7 is primarily described with reference to the components of system 106 as illustrated in FIG. 2, the technique may be performed using any other components of system 106 described herein. While the example technique illustrated in FIG. 7 are primarily described as being performed by a manufacturing assembly, the technique may be performed by a manufacturer and/or by a clinician (e.g., prior to an implantation procedure).

The manufacturing assembly may couple IMD 110 to tether assembly 122 of system 106 (602). Tether assembly 122 may be disposed within an inner lumen of elongated body 109 of delivery catheter 108. The clinician may advance, via proximal handle 126, tether assembly 122 into direction M to cause a tether head assembly of tether assembly 122 to advance at least partially out of distal cup 112 disposed at a distal portion of delivery catheter 108. The clinician may removably couple a proximal end of IID 110 to the tether head assembly of tether assembly 122. For example, the clinician may connect an attachment feature at the proximal end of IMD 110 to the tether head assembly.

The manufacturing assembly may insert IMD 110 into delivery cup 112 of delivery catheter 108 (604). Once TID 110 is coupled to tether assembly 122, the clinician may retract tether assembly 122 into delivery cup 112 (e.g., via retraction of proximal handle 126) to insert IMD 110 into delivery cup 112. Retraction of tether assembly 122 into delivery cup 112 may cause retraction of IID 110 into inner volume 118 of delivery cup 112. When IMD 108 is within inner volume 118, IMD 108 may be maintained in a collapsed configuration, e.g., as illustrated in FIG. 2. For example, when IMD 108 is within inner volume 118, fixation features 114 of IMD 110 may be retained in a collapsed or constrained configuration. The manufacturing assembly may fully retract IMD 108 into inner volume 118 of delivery cup 112, e.g., until a proximal portion of IID 108 contacts a proximal surface within delivery cup 112.

The manufacturing assembly may enclose a distal opening of delivery cup 112 via dissolvable insert 120 (606). Dissolvable insert 120 may be formed from a material configured to dissolve within a body fluid of the patient. Dissolvable insert 120 may include any of dissolvable inserts 302, 402, 502, or any other dissolvable inserts described herein. In some examples, at least a portion of dissolvable insert 120 may be configured to be disposed within inner volume 118. In such examples, a proximal portion of dissolvable insert 120 (e.g., proximal portion 306, 406, 508) may interface with an inner surface of delivery cup 112 to couple dissolvable insert 120 relative to delivery cup 112. The interface between the proximal portion of dissolvable insert 120 and the inner surface of delivery cup 112 may be contact between the two elements, and/or interface between fixation features (e.g., recesses, protrusions) on the two elements. In some examples, once dissolvable insert 120 is inserted into delivery cup 112, dissolvable insert 120 may remain affixed to delivery cup 112 via friction. In some examples, a distal end of dissolvable insert 120 may be flush with a distal end of delivery cup 112 at or around distal end 111A of delivery catheter 108.

In some examples, at least a portion of dissolvable insert 120 may extend distally beyond delivery cup 112. In such examples dissolvable insert 120 may interface with a distal end of delivery cup 112, an inner surface of delivery cup 112, and/or an outer surface of delivery cup 112 to removably couple dissolvable insert 120 to delivery cup 112. In some examples, a distal portion of dissolvable insert 120 (e.g., distal portion 304, 404, 506) may define a distal tip for delivery catheter 108. The distal tip formed by dissolvable insert 120 may be an atraumatic tip.

FIG. 8 is a flow chart illustrating an example technique of implanting a medical device via the medical device system of FIG. 2. While the technique illustrated in FIG. 8 is primarily described with reference to the components of system 106 as illustrated in FIG. 2, the technique may be performed using any other components of system 106 described herein. While the technique illustrated in FIG. 8 is primarily described herein with respect to a target site located in vasculature of a patient (e.g., in a chamber of the heart of the patient), the technique may be used for other target sites within other body lumens of the patient.

A clinician may insert distal end 111A of delivery catheter 108 of system 100 into vasculature of a patient (702). The clinician may form an incision site into the vasculature of the patient. The clinician introduce distal end 111A of delivery catheter 108, including delivery cup 112 at or around distal end 111A, into the vasculature through the incision site. IMD 110 may be loaded into delivery cup 112 of delivery catheter 108, e.g., in a delivery configuration. The distal opening of delivery cup 112 may be enclosed by dissolvable insert 120. Dissolvable insert 120 may be at least partially disposed within inner volume 118 of delivery catheter 108. In such examples, dissolvable insert 120 may be separated from NID 110 (e.g., from distal end 116 of fixation features 114 of IMD 110) while IMD 110 is disposed within inner volume 118 and dissolvable insert 120 encloses the distal opening of delivery cup 112.

The clinician may advance distal end 111A of delivery catheter 108 to a target site within the vasculature (704). As the clinician advances distal end 111A of delivery cup 108 within the vasculature, dissolvable insert 120 may define a distal tip at distal end 111A. The distal tip may be an atraumatic tip. Dissolvable insert 120 may inhibit unintended penetration of wall tissue of the vasculature by delivery catheter 108 and may facilitate travel of distal end 111A of delivery catheter 108 around turns throughout the vasculature. While dissolvable insert 120 is within the vasculature, dissolvable insert 120 may being to dissolve, e.g., due to contact with body fluid within the vasculature. Dissolvable insert 120 may include recesses (e.g., recesses 310, 410) which may control a rate of dissolution of dissolvable insert 120 within the vasculature. Based on a distance between the incision site and the target site, dissolvable insert 120 may be selected such that less than a threshold proportion (e.g., up to 50% up to 75%) of dissolvable insert 120 would dissolve prior to distal end 111A of delivery catheter 108 reaching the target site.

The clinician may advance IMD 110 out of delivery cup 112 of delivery catheter 108 upon at least a partial dissolution of dissolvable insert 120 around a distal opening of delivery cup 112 (706). When distal end 111A of delivery catheter 108 reaches the target site, the clinician may advance IMD 110 out of delivery cup 112 once at least the threshold proportion of dissolvable insert 120 has dissolved. In some examples, when dissolvable insert 120 is completely dissolved, the clinician may advance IMD 110 out of delivery cup 112 via actuation of proximal handle 126 of tether assembly 122.

In some examples, at least the threshold proportion of dissolvable insert 120 has been dissolved, the clinician may introduce, via the inner lumen of elongated body 109, a fluid into the vasculature at or around the target site. The fluid may include, but is not limited to, contrast fluid. The fluid may flow from the inner lumen of elongated body 109, through inner volume 118 of delivery cup 112, and out through the distal opening of delivery cup 112. The flow of fluid through the distal opening of delivery cup 112 may help push a remaining portion of dissolvable insert 120 out of and/or away from delivery cup 112, e.g., thereby facilitating advancement of IMD 110 out of delivery cup 112.

The clinician may affix IMD 110 to tissue at the target site (708). As IMD 110 exits delivery cup 112, fixation features 114 may being to expand (e.g., radially outwards) to assume unconstrained configurations. The clinician may place delivery cup 112 relative to the tissue at the target site such that, as IMD 110 exits delivery cup 112, fixation features 114 expand to puncture the tissue at the target site, thereby affixing IMD 110 to the target site. The target site may be one or more locations within the heart of the patient, e.g., as illustrated in FIG. 1.

Once the clinician determines IMD 110 is successfully affixed to the tissue at the target site, the clinician may release tether assembly 122 from EVID 110 (e.g., via proximal handle 126). The clinician may subsequently retract delivery catheter 108 from within the vasculature of the patient and complete the implantation procedure.

The following examples are illustrative of the techniques described herein.

Example 1: a medical device system comprising: an implantable medical device (IMD); a delivery catheter extending from a proximal end to a distal end, wherein the distal end of the delivery catheter defines a receptacle sized to retain the IMD, the receptacle defining a distal opening; and a dissolvable insert configured to enclose the distal opening of the receptacle, wherein the dissolvable insert is configured to at least partially dissolve within a body fluid of a patient to enable deployment of the IMD from within the receptacle of the delivery catheter.

Example 2. the medical device system of example 1, wherein the dissolvable insert is configured to be disposed over the distal opening, wherein the dissolvable insert defines a distal portion and a proximal portion connected to the distal portion, and wherein when the dissolvable insert is disposed over the distal opening of the receptacle, at least the proximal portion is disposed within the receptacle.

Example 3: the medical device system of example 2, wherein when the proximal portion is disposed within the receptacle, a proximal end of the dissolvable insert is separated from a distal end of the IMD.

Example 4: the medical device system of any of examples 2 or 3, wherein the distal portion defines a first diameter of up to an outer diameter of the distal end of the delivery catheter, and wherein the proximal portion defines a second diameter of up to an inner diameter of the receptacle.

Example 5: the medical device system of example 4, wherein the dissolvable insert comprises one or more protrusions extending from an outer surface of the proximal portion of the dissolvable insert, wherein the one or more protrusions define the second diameter of the proximal portion.

Example 6: the medical device system of any of examples 1-5, wherein the dissolvable insert defines one or more recesses over an outer surface of the distal end of the dissolvable insert.

Example 7: the medical device system of example 6, wherein each recess of the one or more recesses is configured to increase a contact surface area between the dissolvable insert and the body fluid.

Example 8: the medical device of example 7, wherein a rate of dissolution of the dissolvable insert is based at least in part on the contact surface area.

Example 9: the medical device system of any of examples 1-8, wherein the IMD comprises: an elongated housing extending from a proximal end to a distal end; and one or more tines fixation features extending from the distal end of the elongated housing, the one or more tines fixation features being configured to transition between a collapsed configuration and an expanded configuration, wherein the receptacle is configured to retain the one or more tines fixation features in the collapsed configuration, and wherein when the dissolvable insert is configured to enclose the distal opening of the receptacle, the one or more tines fixation features are separated from a proximal end of the dissolvable insert.

Example 10: the medical device system of example 9, wherein the dissolvable insert defines one or more channels over the proximal end of the dissolvable insert, the one or more channels being configured to separate the one or more tines fixation features of the IMD from the dissolvable insert.

Example 11: the medical device system of any of examples 1-10, wherein a proximal end of the dissolvable insert defines a conical shape.

Example 12: the medical device system of any of examples 1-11, wherein a distal end of the dissolvable insert defines one or more of a conical shape, a domed shape, or a substantially flat surface.

Example 13: the medical device system of any of examples 1-12, wherein the IMD comprises an implantable cardiac device.

Example 14: the medical device system of any of examples 1-13, wherein the dissolvable insert comprises a dissolvable material, the dissolvable material comprising a sugar.

Example 15: a dissolvable insert comprising: a distal portion defining a first outer diameter; and a proximal portion connected to the distal portion and defining a second outer diameter, the second outer diameter being different from the first outer diameter, wherein the dissolvable insert is configured to interface with a receptacle of a catheter to at least partially enclose an inner volume of the receptacle, wherein the proximal portion is sized to enter the inner volume of the receptacle, and wherein the distal portion is sized to remain outside of the inner volume of the receptacle, and wherein the dissolvable insert is configured to at least partially dissolve within a body fluid.

Example 16: the dissolvable insert of example 15, wherein the second outer diameter is up to an inner diameter of the inner volume of the receptacle, and wherein the first outer diameter is greater than the inner diameter of the inner volume of the receptacle.

Example 17: the dissolvable insert of any of examples 15 or 16, further comprising one or more protrusions extending from an outer surface of the proximal portion of the dissolvable insert, wherein the one or more protrusions define the second outer diameter of the proximal portion.

Example 18: the dissolvable insert of any of examples 15-17, wherein the distal portion of the dissolvable insert defines a distal surface and one or more recesses extending proximally from the distal surface, wherein each recess of the one or more recesses increases a surface area of the dissolvable insert.

Example 19: the dissolvable insert of any of examples 15-18, wherein the proximal portion of the dissolvable insert defines one or more channels over a proximal end of the dissolvable insert.

Example 20: the dissolvable insert of any of examples 15-19, wherein a proximal end of the dissolvable insert defines a conical shape.

Example 21: the dissolvable insert of any of examples 15-20, wherein a distal end of the dissolvable insert defines one or more of a conical shape, a domed shape, or a substantially flat surface.

Example 22: the dissolvable insert of any of examples 15-21, wherein the dissolvable insert comprises a dissolvable material, the dissolvable material comprising a sugar.

Example 23: a method comprising: disposing an implantable medical device (IMD) within an inner volume of a receptacle at a distal end of a delivery catheter of the medical device system via a distal opening of the receptacle; and securing a dissolvable insert to the distal end of the delivery catheter and over the distal opening of the receptacle to at least partially enclose the inner volume of the receptacle, wherein the dissolvable insert is configured to at least partially dissolve within a body fluid and allow access to the inner volume of the receptacle.

Example 24: the method of example 23, wherein the dissolvable insert comprises a distal portion and a proximal portion, and wherein securing the dissolvable insert to the distal end of the delivery catheter comprises inserting at least the proximal portion of the dissolvable insert into the inner volume of the receptacle via the distal opening.

Example 25: the method of example 24, wherein when at least the proximal portion of the dissolvable insert is disposed within the inner volume of the receptacle, the IMD is separated from the dissolvable insert.

Example 26: the method of any of examples 24 or 25, wherein when at least the proximal portion of the dissolvable insert is disposed within the inner volume of the receptacle, the distal portion of the dissolvable insert defines a distal tip of the delivery catheter.

Example 27: the method of example 26, wherein the distal portion of the dissolvable insert defines an atraumatic tip.

Example 28: the method of any of examples 23-27, wherein the proximal portion of the dissolvable insert defines one or more channels, wherein the IMD comprises: an elongated body extending from a proximal end to a distal end; and one or more protrusions extending from the distal end of the elongated body, and wherein inserting at least the proximal portion of the dissolvable insert into the inner volume of the receptacle comprises causing the one or more protrusions of the IMD to enter the one or more channels of the proximal portion of the dissolvable insert., and wherein the one or more protrusions of the IMD are, when disposed within the one or more channels, separated from the dissolvable insert.

Example 29: a method comprising: advancing, via a delivery catheter of a medical device system, an implantable medical device (IMD) of the medical device system through vasculature of a patient to a target location within the patient, wherein the distal end of the delivery catheter defines a receptacle, wherein the IMD is disposed within an inner volume of the receptacle, and wherein the medical device system further comprises a dissolvable insert disposed over a distal opening of the receptacle to retain the IMD within the receptacle; releasing the dissolvable insert from the receptacle; advancing the IMD distally from within the inner volume of the receptacle; and affixing the IMD to tissue of the patient at the target location.

Example 30: the method of example 29, wherein releasing the dissolvable insert from the receptacle comprises: placing the distal end of the delivery catheter within a body fluid of the patient to cause the dissolvable insert to at least partially dissolve within the body fluid, and wherein advancing the IMD distally from within the inner volume of the receptacle comprises: advancing the IMD out of the inner volume of the receptacle via the distal opening of the receptacle when the dissolvable insert is at least partially dissolved.

Example 31: the method of example 30, wherein the dissolvable insert defines one or more recesses over a distal surface of the dissolvable insert, wherein each recess of the one or more recesses increases a contact surface area between the dissolvable insert and the body fluid.

Example 32: the method of example 31, wherein a rate of dissolution of the dissolvable insert is based at least in part on the contact surface area.

Example 33: the method of any of examples 30-32, wherein the delivery catheter comprises an elongated body defining an inner lumen fluidically coupled to the inner volume of the receptacle, and wherein advancing the IMD distally from within the inner volume of the receptacle when the dissolvable insert is at least partially dissolved comprises: introducing, via the inner lumen of the delivery catheter, a fluid through the receptacle and into the vasculature of the patient, wherein the fluid separates a remaining portion of the dissolvable insert from the receptacle; and once the remaining portion of the dissolvable insert is separated from the receptacle, advancing the IMD distally from within the inner volume of the receptacle.

Example 34: the method of example 33, wherein the fluid comprises a biocompatible contrast medium.

Example 35: the method of any of examples 29-34, wherein the dissolvable insert comprises: a distal portion disposed outside the receptacle; and a proximal portion connected to the distal portion and at least partially disposed within the inner volume of the receptacle.

Example 36: the method of example 35, wherein the distal portion defines a distal tip of the delivery catheter.

Example 37: the method of any of examples 29-36, wherein the IMD comprises an implantable cardiac device.

Example 38: the method of any of examples 29-37, wherein the target location is disposed within a chamber of a heart of the patient.

Example 39: the method of any of examples 29-38, wherein the dissolvable insert comprises a dissolvable material, the dissolvable material comprising a sugar.

Various examples have been described. These and other examples are within the scope of the following claims.

What is claimed is:

1. A medical device system comprising:
an implantable medical device (IMD);
a delivery catheter extending from a proximal end to a distal end, wherein the distal end of the delivery catheter defines a receptacle sized to retain the IMD, the receptacle defining a distal opening; and
a dissolvable insert configured to enclose the distal opening of the receptacle, wherein the dissolvable insert is configured to at least partially dissolve within a body fluid of a patient to enable deployment of the IMD from within the receptacle of the delivery catheter.

2. The medical device system of claim 1, wherein the dissolvable insert is configured to be disposed over the distal opening, wherein the dissolvable insert defines a distal portion and a proximal portion connected to the distal portion, and wherein when the dissolvable insert is disposed over the distal opening of the receptacle, at least the proximal portion is disposed within the receptacle.

3. The medical device system of claim 2, wherein when the proximal portion is disposed within the receptacle, a proximal end of the dissolvable insert is separated from a distal end of the IMD.

4. The medical device system of claim 2, wherein the distal portion defines a first diameter of up to an outer diameter of the distal end of the delivery catheter, and wherein the proximal portion defines a second diameter of up to an inner diameter of the receptacle.

5. The medical device system of claim 4, wherein the dissolvable insert comprises one or more protrusions extending from an outer surface of the proximal portion of the dissolvable insert, wherein the one or more protrusions define the second diameter of the proximal portion.

6. The medical device system of claim 1, wherein the dissolvable insert defines one or more recesses over an outer surface of the distal end of the dissolvable insert.

7. The medical device system of claim 6, wherein each recess of the one or more recesses is configured to increase a contact surface area between the dissolvable insert and the body fluid.

8. The medical device system of claim 1,
wherein the IMD comprises:
an elongated housing extending from a proximal end to a distal end; and
one or more fixation features extending from the distal end of the elongated housing, the one or more fixation features being configured to transition between a collapsed configuration and an expanded configuration,
wherein the receptacle is configured to retain the one or more fixation features in the collapsed configuration, and
wherein when the dissolvable insert is configured to enclose the distal opening of the receptacle, the one or more fixation features are separated from a proximal end of the dissolvable insert.

9. The medical device system of claim 8, wherein the dissolvable insert defines one or more channels over the proximal end of the dissolvable insert, the one or more channels being configured to separate the one or more fixation features of the IMD from the dissolvable insert.

10. The medical device system of claim 1, wherein a distal end of the dissolvable insert defines one or more of a conical shape, a domed shape, or a substantially flat surface.

11. The medical device system of claim 1, wherein the IMD comprises an implantable cardiac device.

12. The medical device system of claim 1, wherein the dissolvable insert comprises a dissolvable material, the dissolvable material comprising a sugar.

13. A dissolvable insert comprising:
a distal portion defining a first outer diameter; and
a proximal portion connected to the distal portion and defining a second outer diameter, the second outer diameter being different from the first outer diameter,
wherein the dissolvable insert is configured to interface with a receptacle of a catheter to at least partially enclose an inner volume of the receptacle, wherein the proximal portion is sized to enter the inner volume of the receptacle, and wherein the distal portion is sized to remain outside of the inner volume of the receptacle, and
wherein the dissolvable insert is configured to at least partially dissolve within a body fluid.

14. The dissolvable insert of claim 13,
wherein the second outer diameter is up to an inner diameter of the inner volume of the receptacle, and
wherein the first outer diameter is greater than the inner diameter of the inner volume of the receptacle.

15. The dissolvable insert of claim 13, further comprising one or more protrusions extending from an outer surface of the proximal portion of the dissolvable insert, wherein the one or more protrusions define the second outer diameter of the proximal portion.

16. The dissolvable insert of claim 13, wherein the distal portion of the dissolvable insert defines a distal surface and one or more recesses extending proximally from the distal surface, wherein each recess of the one or more recesses increases a surface area of the dissolvable insert.

17. The dissolvable insert of claim 13, wherein a distal end of the dissolvable insert defines one or more of a conical shape, a domed shape, or a substantially flat surface.

18. The dissolvable insert of claim 13, wherein the dissolvable insert comprises a dissolvable material, the dissolvable material comprising a sugar.

19. A method comprising:
disposing an implantable medical device (IMD) within an inner volume of a receptacle at a distal end of a delivery catheter of the medical device system via a distal opening of the receptacle; and
securing a dissolvable insert to the distal end of the delivery catheter and over the distal opening of the receptacle to at least partially enclose the inner volume of the receptacle,
wherein the dissolvable insert is configured to at least partially dissolve within a body fluid and allow access to the inner volume of the receptacle.

20. The method of claim 19, wherein the dissolvable insert comprises a distal portion and a proximal portion, and wherein securing the dissolvable insert to the distal end of the delivery catheter comprises inserting at least the proximal portion of the dissolvable insert into the inner volume of the receptacle via the distal opening.
